(19) United States
(12) Patent Application Publication
(10) Pub. No.: US 2015/0065797 A1
(43) Pub. Date: Mar. 5, 2015

(54) ELECTRONIC ENDOSCOPE DEVICE, IMAGING MODULE, AND IMAGE PICK-UP LENS MOLDING METHOD

(71) Applicant: FUJIFILM Corporation, Tokyo (JP)

(72) Inventor: Goki YAMAMOTO, Ashigarakami-gun (JP)

(73) Assignee: FUJIFILM CORPORATION, Tokyo (JP)

(21) Appl. No.: 14/528,436

(22) Filed: Oct. 30, 2014

Related U.S. Application Data
(63) Continuation of application No. PCT/JP2013/061865, filed on Apr. 23, 2013.

(30) Foreign Application Priority Data
May 28, 2012 (JP) 2012-120645

(51) Int. Cl. G02B 23/24; G02B 7/02; A61B 1/05

(52) U.S. Cl. CPC G02B 23/243; A61B 1/05; G02B 7/02
USPC 600/109; 359/819

(57) ABSTRACT

The invention provides an electronic endoscope device, an imaging module, and an image pick-up lens molding method that can prevented dew formation of an objective lens optical system and facilitate manufacture. An objective lens optical system of an imaging module includes a tip lens 50a in which a tip surface and a back surface are formed in a plane, and a recess S is formed; a plane plate 50b that block the recess S; and a lens barrel 51a that integrally molds and forms the entire outer peripheral surfaces of the tip lens 50a and the plane plate 50b with resin, while a state is maintained where the plane plate 50b is pressed against the tip lens 50a, and the plane plate 50b and the back surface of the tip lens 50a are directly brought into close contact with each other at an entire joining surface therebetween.

### CROSS-REFERENCE TO RELATED APPLICATIONS

This application is a Continuation of PCT International Application No. PCT/JP2013/061865 filed on Apr. 23, 2013, which claims priority under 35 U.S.C §119(a) to Japanese Patent Application No. 2012-120645 filed on May 28, 2012. Each of the above application is hereby expressly incorporated by reference, in its entirety, into the present application.

### BACKGROUND OF THE INVENTION

1. Field of the Invention

The present invention relates to an electronic endoscope device, an imaging module, and an image pick-up lens molding method.

2. Description of the Related Art

An imaging module including an imaging element and an objective lens optical system is built in an endoscope tip portion of an electronic endoscope device in such a manner that image light from a part to be observed, which enters through the objective lens optical system, is focused on a light receiving surface of the imaging element.

The objective lens optical system is configured by the combination of a plurality of optical elements, for example, as described in the following JP2010-22617A and JP1997-105871A (JP-H09-105871A). An objective lens optical system described in JP2010-22617A is illustrated in FIG. 6.

A back side of a first optical element G1 that constitutes a tip lens is formed with a spherical recess S that applies lens power to the first optical element G1, and a second optical element G2 that is a plate-shaped member is attached so as to block the recess S. If a sealed state of a gap formed by the recess S is not maintained, dew formation will occur in the recess S and the quality of a captured image will be degraded.

Thus, in the related art, an adhesive layer M is provided on a joining surface between the first optical element G1 and the second optical element G2 to tightly bond both of the elements together so as to maintain the sealed state. However, even if the first optical element G1 and the second optical element G2 are brought into close contact with each other with the adhesive layer M, there is a concern that moisture may permeate into the gap S if a long period of time passes. This concern becomes greater as the length of the adhesive layer M up to the recess S becomes shorter.

As for the present endoscope, the external diameter thereof is about 9 mm and making the diameter smaller is attempted. A light guide through which illumination light is guided, a forceps pipe, and an air/water supply pipe besides the objective lens optical system are provided in an endoscope tip portion. For this reason, the diameter (the diameter D of FIG. 6) of the objective lens optical system becomes about 3 mm to 4 mm, and a bonding margin portion of the adhesive, that is the length of the adhesive layer M will become 1 mm or less,

Moreover, it is difficult to uniformly apply the adhesive to such a narrow place, and there is also a problem in that the assembly cost of the imaging module will be increased. If the adhesive is unevenly applied, a concern becomes high that moisture may permeate into the gap S from an uneven portion of the adhesive. If a surplus adhesive is applied in order to avoid this, the surplus adhesive will ooze out in the direction of an optical axis, and the imaging module will become defective.

For this reason, the problem of the sealability of the recess S of the tip lens should be solved as the objective lens optical system of the imaging module becomes smaller. Moreover, it is necessary to make the assembly of the objective lens optical system easy.

### SUMMARY OF THE INVENTION

An object of the invention is to provide an electronic endoscope device, an imaging module, and an image pick-up lens molding method that can prevent dew formation and facilitate manufacture.

The imaging module of the invention is a lens module including an objective lens optical system; and an imaging element that receives incident light that has entered through the objective lens optical system. The objective lens optical system includes a tip lens in which a tip surface that incident light enters and a back surface opposite to the tip surface are formed in a plane, and a recess that condenses the incident light is formed at a central portion of the back surface; a plane plate that is installed on a back side of the tip lens to block the recess; and a lens barrel that integrally molds and forms the entire outer peripheral surfaces of the tip lens and the plane plate with resin, while a state is maintained where the plane plate is pressed against the tip lens, and the plane plate and the back surface of the tip lens are directly brought into close contact with each other at an entire joining surface therebetween.

The electronic endoscope device of the invention has the above imaging module built in an endoscope tip portion.

The imaging lens molding method of the invention is a method for molding an imaging lens of a lens barrel that houses a tip lens in which a tip surface that incident light enters and a back surface opposite to the tip surface are formed in a plane, and a recess that condenses the incident light is formed at a central portion of the back surface, and a plane plate that is installed on a back side of the tip lens to block the recess. The method includes integrally molding the entire outer peripheral surfaces of the tip lens and the plane plate with resin, while a state is maintained where the plane plate is pressed against the tip lens, and the plane plate and the back surface of the tip lens are directly brought into close contact with each other at an entire joining surface therebetween.

According to the invention, the plane plate and the back surface of the tip lens are directly brought into close contact with each other at the entire surface without providing an adhesive layer therebetween, permeation of moisture into it recess S formed in the tip lens can be prevented, and it is possible to keep the quality of a captured image high.

When an adhesive layer is provided between the plane plate and the back surface of the tip lens, an interface is formed between the plane plate and the adhesive layer, and an interface is also formed between the adhesive layer and the back surface of the tip lens. If the plane plate and the rip lens are bonded together with the adhesive, the entire interfaces of the two interfaces are not brought into a close contact state when the interfaces are viewed from the size of a moisture molecule level, and a gap through which moisture molecules pass will be formed.

In the invention, since no adhesive layer is provided, the number of interfaces, that is, a permeation path for moisture molecules, decreases, and it is possible to prevent permeation of moisture into the recess space S. This effect becomes greater as the imaging module is smaller.

Additionally, since the invention provides the structure in which the adhesive layer is made unnecessary, the assembly of the imaging module becomes easy and it is possible to achieve cost reduction.

### BRIEF DESCRIPTION OF THE DRAWINGS

FIG. 1 is a system configuration view of an electronic endoscope device related to an embodiment of the invention.

FIG. 2 is a perspective view of an endoscope tip portion illustrated in FIG. 1.

FIG. 3 is a cross-sectional schematic view taken along line illustrated in FIG. 2.

FIG. 4 is a view describing a method for manufacturing it preceding stage lens barrel illustrated in FIG. 3.

FIG. 5 is a cross-sectional view of the preceding stage lens barrel and a tip lens manufactured by the method described in FIG. 4.

FIG. 6 is a longitudinal cross-sectional view of an objective lens optical system built in a related-art endoscope tip portion.

### DESCRIPTION OF THE PREFERRED EMBODIMENTS

Hereinafter, an embodiment of the invention will be described with reference to the drawings.

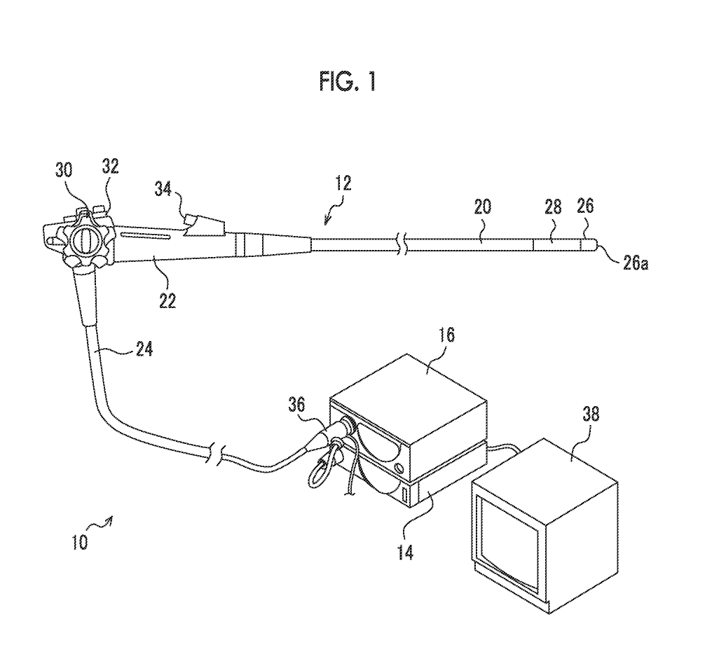

FIG. 1 is a system configuration view of an electronic endoscope device related to an embodiment of the invention. The electronic endoscope device (endoscope system) 10 of the present embodiment is constituted of an endoscope 12, a processor unit 14 and a light source unit 16 that constitute a body device. The endoscope 12 includes a flexible insertion section 20 inserted into a patient's (subject's) body cavity, an operation section 22 provided continuously at a base end portion of the insertion section 20, and a universal cord 24 connected to the processor unit 14 and the light source unit 16.

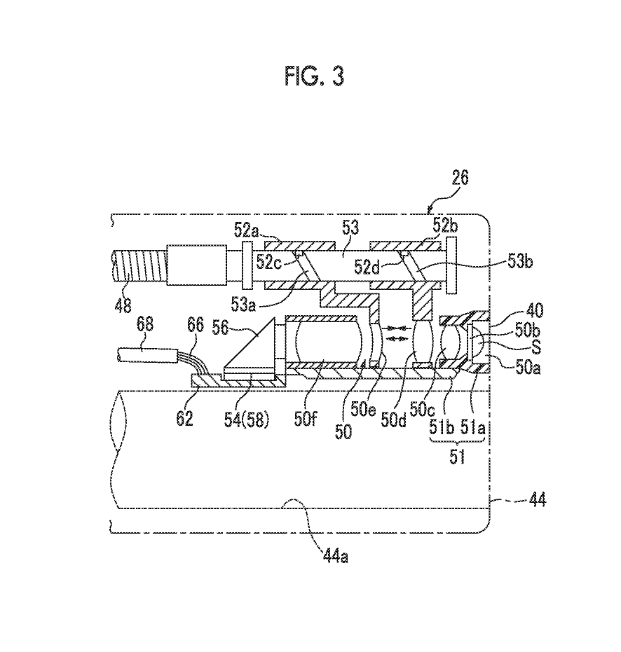

A tip portion 26 is provided continuously at a tip of the insertion section 20, and an imaging chip 54 (refer to FIG. 3) that constitutes an imaging module for picking up an image of the inside of the body cavity is built within the tip portion 26. A bending portion 28 formed by coupling a plurality of bending pieces together is provided behind the tip portion 26. When an angle knob 30 provided at the operation section 22 is operated, a wire inserted into the insertion section 20 is pushed/pulled and the bending portion 2 makes bending motions in vertical and horizontal directions. Accordingly, the tip portion 26 is directed to a desired direction within the body cavity.

A connector 36 is provided at a base end of the universal cord 24. The connector 36 is a composite type connector, and is not only connected to the processor unit 14 but also connected to the light source unit 16.

The processor unit 14 supplies electric power to the endoscope 12 via a cable 68 (refer to FIG. 3) inserted through the universal cord 24 to control the driving of the imaging chip 54, receives imaging signals transmitted via the cable 68 from the imaging chip 54, and performs various signal processing on the received imaging signals to convert the imaging signals to image data.

The image data converted by the processor unit 14 is displayed on as monitor 38 cable-connected to the processor unit 14 as an endoscope pick-up image (observation image). Additionally, the processor unit 14 is also electrically connected to the light source unit 16 via the connector 36, and generally controls the operation of the endoscope system 10 including the light source unit 16.

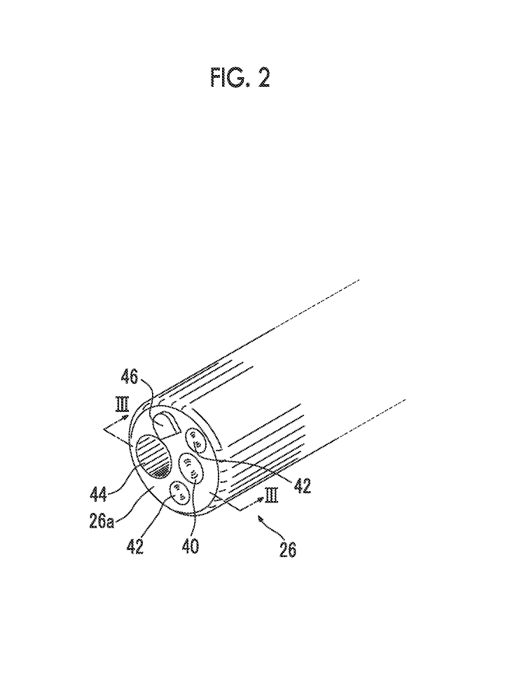

FIG. 2 is a perspective view of the tip portion 26 of the endoscope 12. As illustrated in FIG. 2, a tip surface 26a of the tip portion 26 is provided with an observation window 40, illumination windows 42, a forceps outlet 44, and an air/water supply nozzle 46.

The observation window 40 is arranged so as to be eccentric to one side from center of the tip surface 26a. Two illumination windows 42 are disposed at positions symmetrical to the observation window 40 as a center, and irradiate a part to be observed within the body cavity with illumination light from the light source unit 16.

The forceps outlet 44 is connected to a forceps pipe 44a (refer to FIG. 3) disposed within the insertion section 20, and communicates with a forceps inlet 34 (refer to FIG. 1) provided in the operation section 22. Various treatment tools having an injection needle, a high-frequency knife, and the like disposed at tips thereof are inserted through the forceps inlet 34, and the tips of the various treatment tools are passed into the body cavity from the forceps outlet 44.

The air/water simply nozzle 46 jets washing air or water supplied from an air/water supply unit built in the light source unit 16 toward the observation window 40 or the inside of the body cavity in response to the operation of an air/water supply button 32 (refer to FIG. 1) provided at the operation section 22.

FIG. 3 is a cross-sectional schematic view taken along line III-III of FIG. 2, and is a view illustrating as longitudinal cross-section of the imaging module built in the tip portion 26 of the endoscope 12. As illustrated in FIG. 3, a lens barrel 51 that holds an objective lens optical system 50 for taking in image light of the part to be observed within the body cavity is disposed in the depths of the observation window 40.

The objective lens optical system 50 includes a tip lens 50a, a disc-like transparent parallel plane plate (simply referred to as is plane plate) 50b, a fixed lens 50e, movable lenses 50d and 50e, and a fixed lens 50f from a tip side. Here, the tip side means a tip portion 26 side of the endoscope 12.

An optical axis of the objective lens optical system 50 is provided so as to become parallel to a central axis of the insertion section 20. A prism 56 that bends the image light of the part to be observed, which has been through the objective lens optical system 50, substantially at a right angle and guides the bent image light towards the imaging chip 54 is connected to a rear end of the lens barrel 51.

The imaging chip 54 is constituted by a solid-state imaging element 58, such as a CCD type and a CMOS type, and a monolithic semiconductor with which a peripheral circuit that performs the driving and signal input/output of the signal of the solid-state imaging element 58 are integrally formed. The imaging chip 54 and the peripheral circuit are mounted on a supporting substrate 62. An imaging surface (light receiving surface) of the solid-state imaging element 58 is arranged so as to face an emission surface of the prism 56.

The objective lens optical system 50 in the illustrated example constitutes a zoom lens, and the solid-state imaging element 58 is enabled to capture an image in which the part to be observed is enlarged with a desired magnification by moving the positions of the movable lenses 50d and 50e along the optical axis and changing a mutual distance between the movable lenses or distances from the fixed lenses 50c and 50f.

For this reason, cylindrical cam members 52a and 52b are attached to the movable lenses 50d and 50e, respectively. Inner peripheral surfaces of center holes of the cylindrical cam members 52a and 52b are respectively provided with projections 52c and 52d, and a cam shaft 53 is inserted into the center holes. Cam grooves 53a and 53b that are slidably fitted to the projections 52c and 52d, respectively, are engraved in a peripheral surface of the cam shaft 53.

As the cam shaft 53 is rotationally driven around an axis, the cylindrical cam members 52a and 52b move in an axial direction, and the movable lenses 50d and 50e move along the optical axis of the objective lens optical system 50. The magnifying power of the objective lens optical system 50, that is, the focal distance of the objective lens optical system 50, is adjusted depending on the rotational position of the cam shaft 53.

A power transmission wire 48 is attached to a base end of the cam shaft 53. The power transmission wire 48 is inserted to the operation section 22 of FIG. 1, and is rotationally driven by a motor (not illustrated) provided at the operation section 22. An endoscope operator operates an enlargement/reduction instruction switch of the motor provided at the operation section 22, thereby issuing an instruction for enlargement/reduction of a captured image.

A plurality of input/output terminals are provided side by side on a surface portion of the supporting substrate 62, at a rear end of the supporting substrate 62 provided to extend toward a rear end of the insertion section 20, and signal lines 66 for intermediating exchange of various signals with the processor unit 14 via the universal cord 24 of FIG. 1 are joined to the input/output terminals.

The plurality of signal lines 66 are collectively inserted into the flexible tubular cable 68. The cable 68 is inserted through the insertion section 20, the operation section 22, and the universal cord 24 respectively, and is connected to the connector 36.

Although illustration is omitted in FIGS. 2 and 3, an emission end of a light guide that guides illumination light from the light source unit 16 is disposed in the depths of the illumination window 42. Similar to the cable 68, the light guide configured by bundling a number of optical fibers is inserted through the insertion section 20, the operation section 22, and the universal cord 24, respectively, and an incident end is connected to the connector 36.

The lens barrel 51 of the present embodiment has a two-stage configuration and is constituted by a preceding stage lens barrel 51a and a subsequent stage lens barrel 51b having the same optical axis, and the subsequent stage lens barrel 51b is provided continuously at a rear portion of the preceding stage lens barrel 51a.

The tip lens 50a, the plane plate 50b, and the fixed lens 50c are housed within the preceding stage lens barrel 51a. The movable lenses 50d and 50e and the fixed lens 50f are housed in the subsequent stage lens barrel 51b.

The tip lens 50a is a condensing lens in which a tip surface is planar and a spherical recess S is formed on a back side. The recess S is blocked by the plane plate 50b. A combination structure of the tip lens 50a and the plane plate 50b is similar to the objective lens optical system described in JP2010-22617A.

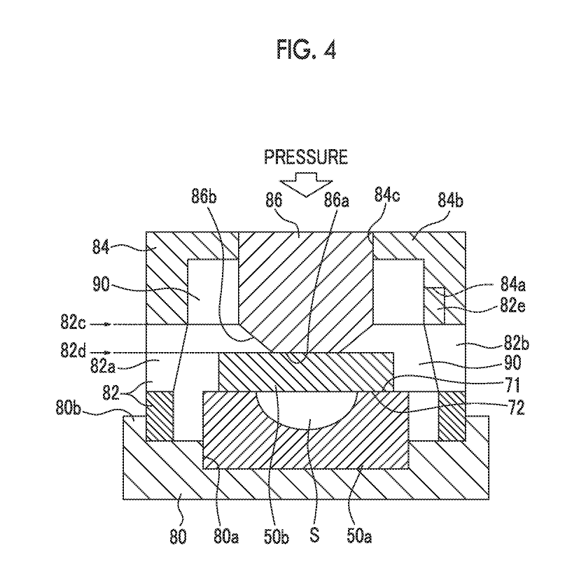

However, the present embodiment provides a structure in which the plane plate 50b is directly brought into close contact with a back surface of the tip lens 50a without providing an adhesive layer between the back surface of the tip lens 50a and the plane plate 50b, and even a slight gap is not formed between a joining surface between both the tip lens and plane plate. This manufacturing method will be described with reference to FIG. 4 to be described below.

When the back surface of the tip lens 50a and the plane plate 50b come into close contact with each other, moisture can be prevented from entering the recess S of the tip lens 50a through the joining surface. That is, even if a cleaning liquid is jetted from the nozzle 46 of FIG. 2 to the tip lens 50a and the temperature of the tip lens 50a falls, a phenomenon in which dew formation occurs within the recess S and the recess space becoming cloudy is prevented.

FIG. 4 is a cross-sectional view illustrating a manufacturing method of providing a structure in which the tip lens 50a made of glass and the plane plate 50b made of glass is molded integrally with the preceding stage lens barrel 51a, the back surface of the tip lens 50a is directly brought into close contact with the plane plate 50b, and even a slight gap is not formed in the joining surface between both the tip lens and the plane plate.

A substantially hemispherical recess S centered on the optical axis is formed in the back surface of the tabular cylindrical tip lens 50a, and a back surface 71 other than the recess S is polished into a flat plane. Although it is better that the flatness of the back surface 71 is higher and it is ideal that the back surface is a perfect plane in which an irregularity difference (height difference) is zero, at least, it is preferable that the polishing roughness is No. 400 or more at a grinding surface.

Although it is better that the flatness of a surface 72 of the plane plate 50b that blocks the recess S of the tip lens 50a is higher, at least, it is also preferable to grind this surface in a plane having the performance of ten or more Newton rings.

Since the external diameters of the tip lens 50a and the plane plate 50b are about 3 mm, it is easy to grind the back surface 71 and the surface 72 with the flatness as described above. In addition, in the illustrated example, the external diameter or the plane plate 50b is made smaller than the external diameter of the tip lens 50a.

A first mold 80 forms a disk shape and a bottomed hole 80a into which a planar tip surface side of the tip lens 50a is drilled at a center position of the first mold. The hole 80a is formed with such an internal diameter that the tip lens 50a is insertable thereinto and a gap is not formed between an outer peripheral surface of the tip lens 50a and an inner peripheral surface of the hole 80a. A central axis of the hole 80a is provided so as to coincide with the optical axis of the tip lens 50a when the tip lens 50a is inserted into the hole 80a.

An annular ring 80b that is concentric with the hole 80a is provided to protrude from an outer peripheral edge of the first mold 80. A cylindrical second mold 82 is fitted to and placed on the first mold 80 so as to come into contact with an inner peripheral surface of the annular ring 80b. When the second mold 82 is fitted to the first mold 80, the second mold 82 becomes concentric with the hole 80a for a tip lens of the first mold 80.

The internal diameter of the second mold 82 is made greater than the external diameter of the tip lens 50a, and is reduced so that the second mold becomes narrow gradually as being away from the hole 80a. Accordingly, a resin tilling space 90 is formed between the second mold 82 and the tip lens 50a. Additionally, radial through-holes 82a and 82b are drilled in a peripheral wall of the second mold 82.

When the second mold 82 is fitted and fixed to the first mold 80, a height position 82c of the second mold 82 has a height above a height position of 82d when the plane plate 50b is placed on the tip lens 50a inserted into the hole 80a.

A third mold 84 placed on the second mold 82 is formed in a cylindrical shape that has an internal diameter that is smoothly continuous with the internal diameter of the second mold 82. A protrusion 82e for alignment is provided to protrude from the second mold 82, and a recess 84a is formed at a position where the recess matches the protrusion 82e, in the third mold 84. That is, when the recess 84a is fitted to the protrusion 82c and the third mold 84 is placed on the second mold 82, the third mold 84 is aligned concentrically with the second mold 82.

A cylindrical opening surface of the third mold 84 opposite to the first mold 80 is blocked by an end wall portion 84b, and a cylindrical through-hole 84c that is coaxial with the hole 80a is drilled at a center position of the end wall portion 84b. The diameter of the through-hole 84c is made greater than the recess S of the tip lens 50a and is made smaller than the external diameter of the plane plate 50b.

A fourth mold 86 inserted into the through-hole 84c of the third mold 84 forms a columnar shape, and a tip surface 86a that presses the plane plate 50b against the tip lens 50a side within the first, second, and third molds 80, 82, and 84, respectively, is formed in a plane. The external diameter of the fourth mold 86 is made substantially equal to the internal diameter of the through-hole 84c in such a manner that a gap is not formed between both the fourth mold and the through hole. A tip portion of the fourth mold 86 is formed at an inclined portion 86b in which a columnar corner is chamfered and is reduced in diameter toward a tip surface. The external diameter of the tip surface 86a is reduced so as to have a smaller diameter than the diameter of the recess S of the tip lens 50a.

As described with reference to FIG. 4, while the tip lens 50a is installed in the hole 80a of the first mold 80, the plane plate 50b is placed on the tip lens 50a. Then, the second mold 82 is placed on the first mold 80 so as to be concentrical with the first mold, the third mold 84 is placed on the second mold so as to be concentrical with the second mold, and finally, the fourth mold 86 is inserted.

Since the plane plate 50b does not have a condensing action, an optical axis is not present. For this reason, there is no problem even if the central axis of the plane plate 50b slightly shifts with respect to the optical axis. Then, the fourth mold 86 is pressed, the plane plate 50b is pressed against the back surface of the tip lens 50a, and a space 90 inside the dies is filled with molding resin from the lateral wall openings $2a and 82b of the second mold 82 while mechanically keeping the close contact state between both the plane plate and the tip lens.

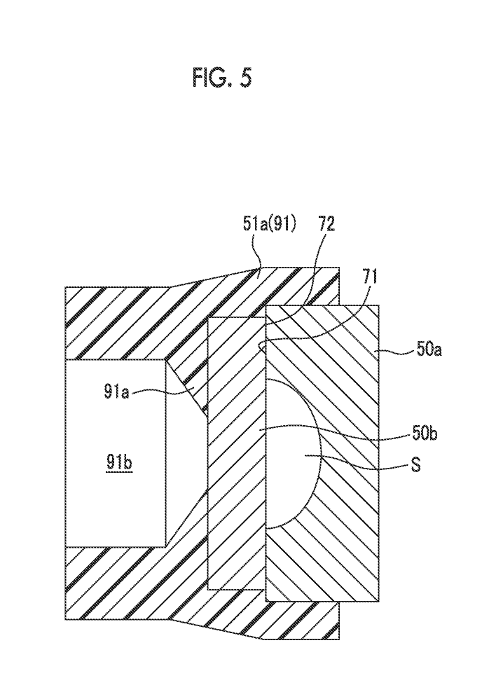

After the molding resin is cured, the first to fourth molds 80, 82, 84, and 86, respectively, are removed, and burrs of the resin and the resin within the openings 82a and 82b are removed. Accordingly, an integral structural article of the first lens barrel 51a and the tip lens 50a, and the plane plate 50b is completed. A longitudinal cross-sectional view of this integral structural article is illustrated in FIG. 5.

In FIG. 5, molding resin 91 cured in a cylindrical shape constitutes the preceding stage lens barrel 51a of FIG. 3. A tip portion of the resin 91 covers about ⅔ of the base end side in the outer peripheral surface of the tip lens 51a. Since the liquid resin 91 is made to flow into the space 90 (refer to FIG. 4) and is cured, the resin 91 and the peripheral wall surface of the tip lens 50a are brought into a close contact state and an anchored state similar to when being bonded with an adhesive.

The resin 91 covers the entire surface of the outer peripheral surface of the plane plate 50b and is brought into close contact with and anchored to the outer peripheral surface, and covers most of a back-side periphery of the plane plate 50b and is brought into close contact with and anchored to the back-side periphery. A flange portion 91a provided to protrude in the direction of an inner periphery of the resin 91 that covers the back side of the plane plate 50b is a portion formed by the inclined portion 86b of the fourth mold 86 of FIG. 4.

Since a circular tip surface of the fourth mold 86 has a smaller diameter than the diameter of a back surface recess S of the tip lens 50a the resin (flange portion) 91 has such a shape that the resin covers all of the joining surface between the back surface 71 of the tip lens 50a and the surface 72 of the plane plate 50b.

When the preceding stage lens barrel 51a is manufactured, the fourth mold 86 of FIG. 4 is pressed against the plane plate 50b at a predetermined pressure or higher, and the resin 91 is made to flow into the space 90 and cured while maintaining this state.

If only the plane plate 50 is pressed down to such a degree that the plane plate does not shift from the tip lens 50a without pressing the mold 86 at a predetermined pressure or higher, and the resin 91 is made to flow in and cured, a gap will be formed in the joining surface between the back surface 71 and the surface 72. This is because, even if the resin 91 does not flow in between the back surface 71 and the surface 72, a gap of about 1 micron will be formed if the irregularity difference between the respective surfaces 71 and 72 is present at about 1 micron. Although it can be said that this gap is small, it is sufficient for moisture to permeate into the recess S.

Thus, in the present embodiment, the method of pressing the mold 86 against the plane plate 50 at a predetermined pressure or higher and making the resin 91 flow into the molds to cure the resin is adopted. The amount of pressure the mold is pressed depends on the material or thickness of the plane plate 50 and the flatness of each of the surfaces 71 and 72 in the joining surface. If the flatness is made high, the plane plate 50 can be only slightly deflected in such a manner that the respective surfaces 71 and 72 in the joining surface are brought into close contact with each other over their entire surfaces with a low pressure, and even a slight gap can be prevented from being formed. With this state maintained, the resin 91 is made to flow into the molds and cured.

The resin 91 is cured in a state where the flange 91a of the resin 91 covers the entire surface of the joining surface on which the back surface 71 of the tip lens 50a and the surface 72 of the plane plate 50 overlap each other and the plane plate 50b is pressed against the tip lens 50a side. Accordingly, even if the preceding stage lens barrel 51a is removed from the molds, the close contact between the plane plate 50b and the tip lens 50a is held at the joining surface therebetween.

In addition, the fixed lens 50c of FIG. 3 is attached within the space 91b from which the mold 86 of the preceding stage lens barrel in was removed. Then, the preceding stage lens barrel 51a and the subsequent stage lens barrel 51b are coupled together so as to have the same optical axis, thereby completing the objective lens optical system 50. Then, an imaging module for an endoscope is completed as the prism 56 and the imaging chip 54 (and substrate 62) are connected to the objective lens optical system 50.

As described above, according to the imaging module related to the embodiment, permeation of moisture into the recess space S formed in the tip lens can be prevented, and it is possible to keep the quality of a captured image high. Additionally, since no adhesive layer is provided between the tip lens 50a and the plane plate 50b, malfunctions caused by the physical and chemical degradation of the adhesive layer can also be prevented. Moreover, by virtue of the structure in which the adhesive layer is made unnecessary, the assembly of the imaging module becomes easy and it is possible to achieve cost reduction.

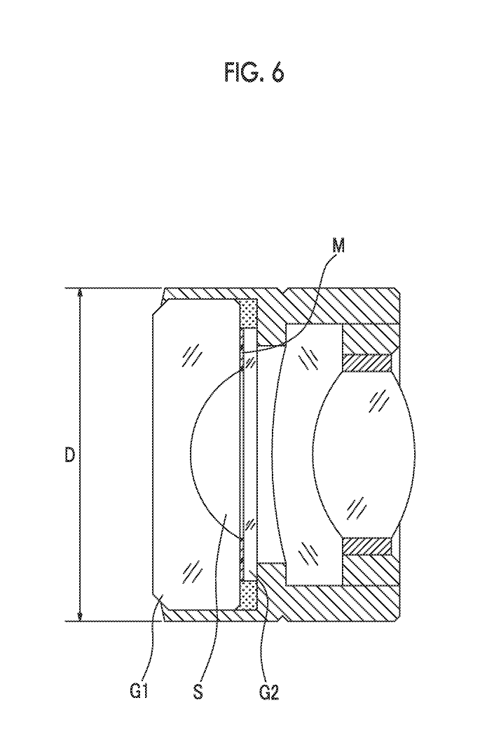

In addition, although the embodiment illustrated in FIG. 5 is configured so that the tip of the resin 91 extends up to a mid portion of the peripheral wall of the tip lens 50a, for example as in the related art of FIG. 6, the tip of the resin may extend to the tip surface of the tip lens and a hook portion may be formed on the tip portion so as to pinch the tip lens 50a.

Additionally, although the imaging module for an endoscope has been described in the above-described embodiment, for example, the invention can also be similarly applied to an imaging module in which the diameter of the objective lens optical system is small, as in an imaging module built in a mobile telephone with a built-in camera or the like.

As described above, the imaging module of the embodiment is an imaging molding including an objective lens optical system, and an imaging element that receives incident light that has entered through the objective lens optical system. The objective lens optical system includes a tip lens in which a tip surface that incident light enters and a back surface opposite to the tip surface are formed in a plane, and a recess that condenses the incident light is formed at a central portion of the back surface; a plane plate that is installed on a back side of the tip lens to block the recess, and a lens barrel that integrally molds and forms the entire outer peripheral surfaces of the tip lens and the plane plate with resin, while a state is maintained where the plane plate is pressed against the tip lens, and the plane plate and the back surface of the tip lens are directly brought into close contact with each other at an entire joining surface therebetween.

Additionally, in the imaging module of the embodiment, the external diameter of the plane plate is smaller than the external diameter of the tip lens.

Additionally, in the imaging module of the embodiment, the entire outer peripheral surfaces of the tip lens and the plane plate and a peripheral region that is a non-passage for the incident light in the back surface of the plane plate are integrally molded with resin.

Additionally, in the imaging module of the embodiment, the peripheral region is a region that covers the entire surface of the joining surface.

Additionally, the electronic endoscope device of the embodiment has the above imaging module built in an endoscope tip portion.

Additionally, the imaging lens molding method of the embodiment is a method for molding an imaging lens of a lens barrel that houses a tip lens in which a tip surface that incident light enters and a back surface opposite to the tip surface are formed in a plane, and a recess that condenses the incident light is formed at a central portion of the back surface, and a plane plate that is installed on a back side of the tip lens to block the recess. The method includes integrally molding the entire outer peripheral surfaces of the tip lens and the plane plate with resin, while a state is maintained where the plane plate is pressed against the tip lens, and the plane plate and the back surface of the tip lens are directly brought into close contact with each other at an entire joining surface therebetween.

According to the embodiment described above, since the back surface of the tip lens and the plane plate are mechanically brought into close contact with each other at the entire joining surface therebetween without using an adhesive, it is possible to capture a high-quality image while being resistant to moisture. Additionally, since no adhesive is used, assembly becomes easy.

Since the tip lens portion of the imaging module related to the invention is resistant to moisture, the invention is useful when built in an imaging device used under a humid environment, especially an endoscope tip portion.

What is claimed is:

1. An imaging module comprising:
an objective lens optical system; and
an imaging element that receives incident light that has entered through the objective lens optical system,
wherein the objective lens optical system includes:
a tip lens in which a tip surface that incident light enters and a back surface opposite to the tip surface are formed in a plane, and a recess that condenses the incident light is formed at a central portion of the back surface;
a plane plate that is installed on a back side of the tip lens to block the recess; and
a lens barrel that integrally molds and forms the entire outer peripheral surfaces of the tip lens and the plane plate with resin, while a state is maintained where the plane plate is pressed against the tip lens, and the plane plate and the back surface of the tip lens are directly brought into close contact with each other at an entire joining surface therebetween.

2. The imaging module according to claim 1,
wherein the external diameter of the plane plate is smaller than the external diameter of the tip lens.

3. The imagine module according to claim 1,
wherein the entire outer peripheral surfaces of the tip lens and the plane plate and a peripheral region that is a non-passage for the incident light in the back surface of the plane plate are integrally molded with resin.

4. The imaging module according to claim 2,
wherein the entire our peripheral surfaces of the tip lens and the plane plate and a peripheral region that is a non-passage for the incident light in the back surface of the plane plate are integrally molded with resin.

5. The imaging module according to claim 3,
the peripheral region is a region that covers the entire surface of the joining surface.

6. The imaging module according to claim 4,
the peripheral region is a region that covers the entire surface of the joining surface.

7. An electronic endoscope device having the imaging module according to claim 1 built in an endoscope tip portion.

8. An electronic endoscope device having the imaging module according to claim 2 built in an endoscope tip portion.

9. An electronic endoscope device having the imaging module according to claim 3 built in an endoscope tip portion.

10. An electronic endoscope device having the imaging module according to claim 4 built in an endoscope tip portion.

11. An electronic endoscope device having the imaging module according to claim 5 built in an endoscope tip portion.

12. An electronic endoscope device having the imaging module according to claim 6 built in an endoscope tip portion.

13. A method for molding an imaging lens of a lens barrel that houses a tip lens in which a tip surface that incident light enters and a back surface opposite to the tip surface are formed in a plane, and a recess that condenses the incident light is formed at a central portion of the back surface, and a plane plate that is installed on a back side of the tip lens to block the recess, the method comprising:
integrally molding the entire outer peripheral surfaces of the tip lens and the plane plate with resin, while a state is maintained where the plane plate is pressed against the tip lens, and the plane plate and the back surface of the tip lens are directly brought into close contact with each other at an entire joining surface therebetween.
